(19) United States
(12) Patent Application Publication
(10) Pub. No.: US 2025/0235471 A1
(43) Pub. Date: Jul. 24, 2025

(54) OIL-IN-WATER EMULSION FOR INHALATION ADMINISTRATION COMPRISING CANNABIDIOL(CBD)

(71) Applicant: Incannex Healthcare Limited, Norwest, New South Wales (AU)

(72) Inventors: Nicholas Dean Childerhouse, Wiltshire (GB); Neil Liam Andrew Forbes, Wiltshire (GB); Mireia Puig Sellart, Wiltshire (GB); Mark Robert Bleackley, Victoria (AU)

(21) Appl. No.: 18/698,191

(22) PCT Filed: Oct. 7, 2022
(86) PCT No.: PCT/AU2022/051200

§ 371 Date: Apr. 3, 2024

(30) Foreign Application Priority Data
Oct. 7, 2021 (AU) 2021903210

(51) Int. Cl. A61K 31/00; A61K 9/00; A61K 9/107; A61K 31/08; A61K 47/44

(52) U.S. Cl. CPC A61K 31/658; A61K 9/0078; A61K 9/107; A61K 31/08; A61K 47/44

(57) ABSTRACT

The present invention relates to a pharmaceutical composition comprising an oil-in-water emulsion having a dispersed oil phase and a continuous water phase, wherein the dispersed oil phase comprises cannabidiol (CBD) or a pharmaceutically acceptable salt or functional derivative thereof, wherein the composition is formulated for administration by inhalation. The composition is intended for use in the treatment of traumatic brain injury (TBI).

### RELATED APPLICATIONS

This application claims priority from Australian Provisional Patent Application No. 2021903210 filed on 7 Oct. 2021, the entire content of which is hereby incorporated by reference.

### FIELD

The present disclosure relates generally to compositions comprising cannabidiol (CBD), which are formulated for administration by inhalation.

### BACKGROUND

Cannabidiol (CBD) is a major cannabinoid constituent of Cannabis species, which is used for the treatment of a variety of different indications. Purified CBD has been approved for use in the treatment of rare, pediatric forms of epilepsy, with on-going clinical trials assessing the use of CBD for the treatment of anxiety, schizophrenia, addiction, post-traumatic stress disorder, graft-versus-host disease, cancer and inflammatory bowel disease.

Although CBD has been considered for the treatment of a broad range of indications, the effective delivery of CBD via different administrative routes has proven to be difficult. Successful drug delivery is dependent on multiple factors, including the patient's physiology and the drug's physiochemical properties (e.g., solubility, dissolution, stability, permeability and metabolism). In this context, the highly lipophilic nature of CBD, with an estimated oil/water partition coefficient (Log P) of 6-7, results in very poor aqueous solubility (<10 μg/mL), which has limited pharmaceutical CBD formulations to oil or alcohol-based formulations for oral administration (e.g., soft-gel capsules, liquid solutions, sublingual drops). However, studies examining oral delivery of CBD in humans demonstrate high inter/intra-individual variability. Further, while CBD has some solubility within specific solvents, such as ethanol, methanol and DMSO, high concentrations of these solvents cannot be delivered to pulmonary tissue.

Alternative administrative routes such as smoking or vaporization can overcome some of the problems associated with CBD bioavailability. However, smoking and vaporization of CBD increases symptoms of respiratory irritation and has the potential to alter lung function. Therefore, there remains an urgent need for the development of compositions comprising CBD, which are formulated for administration by inhalation.

### BRIEF DESCRIPTION OF THE DRAWINGS

Embodiments of the disclosure are described herein, by way of non-limiting example only, with reference to the accompanying drawings.

FIG. 1 shows the effect of high pressure homogenization cycles (x-axis) on oil droplet size (left y-axis; nm) and polydispersity index (right y-axis; PDI).

### SUMMARY

In an aspect of the present disclosure, there is provided a pharmaceutical composition comprising an oil-in-water emulsion having a dispersed oil phase and a continuous water phase, wherein the dispersed oil phase comprises cannabidiol (CBD) or a pharmaceutically acceptable salt or functional derivative thereof, wherein the composition is formulated for administration by inhalation.

In another aspect of the present disclosure, there is provided a nebulized pharmaceutical composition comprising an oil-in-water emulsion having a dispersed oil phase and a continuous water phase, wherein the dispersed oil phase comprises CBD or a pharmaceutically acceptable salt or functional derivative thereof.

In another aspect of the present disclosure, there is provided a method for the treatment or prevention of TBI comprising administering a therapeutically effective amount of the pharmaceutical composition or nebulized pharmaceutical composition described herein to a subject in need thereof.

In another aspect of the present disclosure, there is provided a use of an oil-in-water emulsion having a dispersed oil phase and a continuous water phase in the manufacture of a medicament for the treatment or prevention of TBI, wherein the dispersed oil phase comprises CBD or a pharmaceutically acceptable salt or functional derivative thereof, wherein the medicament is formulated for administration by inhalation.

### DETAILED DESCRIPTION

Unless defined otherwise, all technical and scientific terms used herein have the same meaning as commonly understood by those of ordinary skill in the art to which the disclosure belongs. Any materials and method similar or equivalent to those described herein can be used to practice the present invention.

Throughout this specification, unless the context requires otherwise, the word “comprise”, or variations such as “comprises” or “comprising”, will be understood to imply the inclusion of the stated element or integer or group of elements or integers but not the exclusion of any other element or integer or group of elements or integers.

The phrase “consisting of” means including, and limited to, whatever follows the phrase “consisting of”. Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a compound” includes a single compound, as well as two or more compounds; reference to “an agent” includes one agent, as well as two or more agents; and so forth.

The term “about” will be understood by persons skilled in the art and will vary to some extent depending on the context in which it is used. If there are uses of the term that are not clear to persons skilled in the art, given the context which it is used, “about” will mean up to plus or minus 10% of the particular value.

The present disclosure is predicated, at least in part, on the inventors' surprising finding that pharmaceutical compositions comprising cannabidiol (CBD) can be formulated for administration by inhalation. In an embodiment, by providing the pharmaceutical compositions by nebulization or nasal delivery, nanoscale oil droplets may be delivered to pulmonary tissues.

Thus, in an aspect disclosed herein, there is provided a pharmaceutical composition comprising an oil-in-water emulsion having a dispersed oil phase and a continuous water phase, wherein the dispersed oil phase comprises CBD or a pharmaceutically acceptable salt or functional derivative thereof, wherein the composition is formulated for administration by inhalation.

### Cannabidiol (CBD)

The terms “cannabidiol” or “CBD” are used interchangeably herein to refer to a cannabinoid produced by plants of the genus Cannabis. CBD has antagonist activity on agonists of the CB1 and CB2 receptors and acts as an inverse agonist of the CB1 and CB2 receptors.

CBD is synthesized in cannabis plants as cannabidiolic acid (CBDA), which decarboxylates to CBD (Table 1). While some decarboxylation may occur in the plant, decarboxylation typically occurs post-harvest and is increased by exposing plant material to heat (Sanchez and Verpoote, 2008, Plant Cell Physiology, 49 (12): 1767-82). Decarboxylation is usually achieved by drying and/or heating the plant material. Persons skilled in the art would be familiar with methods by which decarboxylation of CBDA can be promoted, illustrative examples of which include air-drying, combustion, vaporization, curing, heating and baking. The decarboxylated CBD will typically bind to and/or stimulate, directly or indirectly, cannabinoid receptors including CB1 and/or CB2.

CBD may be extracted from any suitable plant parts including leaves, flowers or stems and may be produced by any suitable means known to those skilled in the art. For example, CBD extracts may be produced by extraction with supercritical or subcritical CO₂, or by volatilization of plant material with a heated gas. Illustrative examples of methods used to extract CBD and other cannabinoids from plant material include the methods described in U.S. Pat. No. 10,189,762 and WO 2004/016277.

In an embodiment, the CBD is a synthetic CBD.

CBD is a chiral compound, although only the (−) CBD enantiomer is present in cannabis plants.

The term “enantiomer” as used herein refers to asymmetric molecules that can exist in two different isomeric forms, which have different configurations in space. An enantiomer can rotate plane-polarized light and is, therefore, optically active. Two different enantiomers of the same compound will rotate plane-polarized light in the opposite direction, thus the light can be rotated to the left or counterclockwise for a hypothetical observer (i.e., “levorotatory” or “−”) or it can be rotated to the right or clockwise (i.e., “dextrorotatory” or “+”).

In an embodiment, the synthetic CBD is a racemic mixture, comprising the (−) CBD enantiomer and the (+) CBD enantiomer.

In an embodiment, the synthetic CBD consists of the (−) CBD enantiomer.

The present disclosure contemplates the use of pharmaceutically acceptable salts of CBD. Suitable pharmaceutically acceptable salts of CBD would be known to persons skilled in the art, illustrative examples of which include salts or esters prepared from pharmaceutically acceptable non-toxic bases or acids, including inorganic bases or acids and organic bases or acids, which would be known to persons skilled in the art.

The present disclosure further contemplates the use of functional derivatives of CBD. Suitable functional derivatives of CBD would be known to persons skilled in the art, illustrative examples of which include 7-OH-CBD (7-hydrocannabidiol), methoxylated CBD derivatives (e.g., CBDM, or 2-methoxycannabidiol and CBDD, or 2,6-dimethoxycannabidiol), cannabidiorcol (CBD-C₁) and the CBD derivatives described by Morales et al. (2017, Frontiers in Pharmacology, 8:422).

### Pharmaceutical Compositions

For inhalation, the pharmaceutical compositions can be formulated for administration by any suitable delivery system known to persons skilled in the art, illustrative examples of which include liquid delivery systems, MDI, nebulizers, propellant systems, nasal sprays and the like.

CBD is a highly lipophilic molecule, with an estimated Log P value of 6-7. Accordingly, in order to achieve adequate dissolution of CBD into a liquid, oils and/or lipids are utilized to prepare the dispersed phase. As such, the terms “dispersed phase” and “oil phase” and “dispersed oil phase” may be used interchangeably herein.

In an embodiment, the pharmaceutical composition comprises from about 1 mg/mL to about 1000 mg/mL CBD or a pharmaceutically acceptable salt or functional derivative thereof (e.g., 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, 300 mg/mL, 310 mg/mL, 320 mg/mL, 330 mg/mL, 340 mg/mL, 350 mg/mL, 360 mg/mL, 370 mg/mL, 380 mg/mL, 390 mg/mL, 400 mg/mL, 410 mg/mL, 420 mg/mL, 430 mg/mL, 440 mg/mL, 450 mg/mL, 460 mg/mL, 470 mg/mL, 480 mg/mL, 490 mg/mL, 500 mg/mL, 510 mg/mL, 520 mg/mL, 530 mg/mL, 540 mg/mL, 550 mg/mL, 560 mg/mL, 570 mg/mL, 580 mg/mL, 590 mg/mL, 700 mg/mL, 710 mg/mL, 720 mg/mL, 730 mg/mL, 740 mg/mL, 750 mg/mL, 760 mg/mL, 770 mg/mL, 780 mg/mL, 790 mg/mL, 800 mg/mL, 810 mg/mL, 820 mg/mL, 830 mg/mL, 840 mg/mL, 850 mg/mL, 860 mg/mL, 870 mg/mL, 880 mg/mL, 890 mg/mL, 900 mg/mL, 910 mg/mL, 920 mg/mL, 930 mg/mL, 940 mg/mL, 950 mg/mL, 960 mg/mL, 970 mg/mL, 980 mg/mL, 990 mg/mL, or 1000 mg/mL CBD or a pharmaceutically acceptable salt or functional derivative thereof).

Thus, in an embodiment, the pharmaceutical composition comprises from about 1 mg/mL to about 1000 mg/mL CBD or a pharmaceutically acceptable salt or functional derivative thereof, preferably about 1 mg/mL, preferably about 2 mg/mL, preferably about 3 mg/mL, preferably about 4 mg/mL, preferably about 5 mg/mL, preferably about 6 mg/mL, preferably about 7 mg/mL, preferably about 8 mg/mL, preferably about 9 mg/mL, preferably about 10 mg/mL, preferably about 20 mg/mL, preferably about 30 mg/mL, preferably about 40 mg/mL, preferably about 50 mg/mL, preferably about 60 mg/mL, preferably about 70 mg/mL, preferably about 80 mg/mL, preferably about 90 mg/mL, preferably about 100 mg/mL, preferably about 110 mg/mL, preferably about 120 mg/mL, preferably about 130 mg/mL, preferably about 140 mg/mL, preferably about 150 mg/mL, preferably about 160 mg/mL, preferably about 170 mg/mL, preferably about 180 mg/mL, preferably about 190 mg/mL, preferably about 200 mg/mL, preferably about 210 mg/mL, preferably about 220 mg/mL, preferably about 230 mg/mL, preferably about 240 mg/mL, preferably about 250 mg/mL, preferably about 260 mg/mL, preferably about 270 mg/mL, preferably about 280 mg/mL, preferably about 290 mg/mL, preferably about 300 mg/mL, preferably about 310 mg/mL, preferably about 320 mg/mL, preferably about 330 mg/mL, preferably about 340 mg/mL, preferably about 350 mg/mL, preferably about 360 mg/mL, preferably about 370 mg/mL, preferably about 380 mg/mL, preferably about 390 mg/mL, preferably about 400 mg/mL, preferably about 410 mg/mL, preferably about 420 mg/mL, preferably about 430 mg/mL, preferably about 440 mg/mL, preferably about 450 mg/mL, preferably about 460 mg/mL, preferably about 470 mg/mL, preferably about 480 mg/mL, preferably about 490 mg/mL, preferably about 500 mg/mL, preferably about 510 mg/mL, preferably about 520 mg/mL, preferably about 530 mg/mL, preferably about 540 mg/mL, preferably about 550 mg/mL, preferably about 560 mg/mL, preferably about 570 mg/mL, preferably about 580 mg/mL, preferably about 590 mg/mL, preferably about 700 mg/mL, preferably about 710 mg/mL, preferably about 720 mg/mL, preferably about 730 mg/mL, preferably about 740 mg/mL, preferably about 750 mg/mL, preferably about 760 mg/mL, preferably about 770 mg/mL, preferably about 780 mg/mL, preferably about 790 mg/mL, preferably about 800 mg/mL, preferably about 810 mg/mL, preferably about 820 mg/mL, preferably about 830 mg/mL, preferably about 840 mg/mL, preferably about 850 mg/mL, preferably about 860 mg/mL, preferably about 870 mg/mL, preferably about 880 mg/mL, preferably about 890 mg/mL, preferably about 900 mg/mL, preferably about 910 mg/mL, preferably about 920 mg/mL, preferably about 930 mg/mL, preferably about 940 mg/mL, preferably about 950 mg/mL, preferably about 960 mg/mL, preferably about 970 mg/mL, preferably about 980 mg/mL, preferably about 990 mg/mL, or more preferably about 1000 mg/mL of CBD or a pharmaceutically acceptable salt or functional derivative thereof.

In an embodiment, the pharmaceutical composition comprises from about 1 mg/mL to about 100 mg/mL CBD or a pharmaceutically acceptable salt or functional derivative thereof. In another embodiment, the pharmaceutical composition comprises about 10 mg/mL CBD or a pharmaceutically acceptable salt or functional derivative thereof.

In an embodiment, the pharmaceutical composition comprises from about 0.5% (w/v) to about 5% (w/v) CBD or a pharmaceutically acceptable salt or functional derivative thereof (e.g., about 0.5% (w/v), about 0.6% (w/v), about 0.7% (w/v), about 0.8% (w/v), about 0.9% (w/v), about 1% (w/v), about 2% (w/v), about 3% (w/v), about 4% (w/v) or about 5% (w/v) CBD or a pharmaceutically acceptable salt or functional derivative thereof).

In an embodiment, the pharmaceutical composition comprises from about 0.5% (w/v) to about 2% (w/v) CBD or a pharmaceutically acceptable salt or functional derivative thereof. In another embodiment, the pharmaceutical composition comprises about 1% (w/v) CBD or a pharmaceutically acceptable salt or functional derivative thereof.

In an embodiment, the pharmaceutical composition further comprises one or more pharmaceutically acceptable carriers, diluents and excipients.

In an embodiment, the pharmaceutically acceptable excipients are selected from the group consisting of polyethylene glycol, ethanol, sodium chloride, sodium citrate, tris(hydroxymethyl)aminomethane (TRIS), citric acid (anhydrous), ethylenediaminetetraacetic acid (EDTA), ascorbic acid, sodium metabilsulfite and benzalkonium chloride.

Persons skilled in the art will appreciate that the term “excipient” as used herein encompasses a number of different classes of compounds. For example, the term “excipient” as used herein is intended to encompass solvents, which would be known to persons skilled in the art, illustrative examples of which include dimethylsulfoxide (DMSO), ethanol and benzyl alcohol. In some embodiments, the pharmaceutical composition described herein comprises two or more different solvents, also referred to herein as “co-solvents”. The terms “co-solvent” and “excipient” can be used interchangeably herein.

In an embodiment, the dispersed oil phase comprises an oil selected from the group consisting of sesame oil, olive oil, coconut oil, medium chain triglyceride (MCT) oil and oleic acid. In a preferred embodiment, the oil is oleic acid.

It is also contemplated herein that the pharmaceutical composition comprises surfactants and/or co-solvents.

The term “surfactant” as used herein refers to any compound that lowers the surface tension between two liquids or between a liquid and a solid. Surfactants are amphiphilic, that is, they comprise hydrophilic and hydrophobic parts. Suitable surfactants would be known to persons skilled in the art, illustrative examples of which include emulsifiers (e.g., polyglycerol polyricinoleate (PGPR), sorbitan monooleate (Span 80), lecithin) and detergents (e.g., polysorbate 20, ceteareth 20).

In an embodiment, the pharmaceutical composition further comprises at least one surfactant selected from the group consisting of polysorbate 80 (Tween 80), sorbitan trioleate (Span 85), disaturated-phosphatidylcholine (DSPC), and lecithin.

In an embodiment, the surfactant is Tween 80.

In an embodiment, the surfactant is Tween 80 and Span 85. In another embodiment, the pharmaceutical composition comprises Tween 80 and Span 85 at a ratio of from about 1:1 to about 10:1 (Tween 80:Span 85; e.g., 1:1, 2:1, 3:1, 4:1, 5:1, 6:1, 7:1, 8:1, 9:1, or 10:1).

Thus, in an embodiment, the pharmaceutical composition comprises Tween 80 and Span 85 at a ratio of from about 1:1 to about 10:1 (Tween 80:Span 85), preferably about 1:1, preferably about 2:1, preferably about 3:1, preferably about 4:1, preferably about 5:1, preferably about 6:1, preferably about 7:1, preferably about 8:1, preferably about 9:1, or more preferably about 10:1.

In a preferred embodiment, the pharmaceutical composition comprises Tween 80 and Span 85 at a ratio of about 3:1 (Tween 80:Span 85).

In an embodiment, the pharmaceutical composition comprises Tween 80 and lecithin. In another embodiment, wherein the pharmaceutical composition comprises Tween 80 and lecithin at a ratio of from about 5:1 to about 20:1 (Tween 80:lecithin; e.g., 5:1, 6:1, 7:1, 8:1, 9:1, 10:1, 11:1, 12:1, 13:1, 14:1, 15:1, 16:1, 17:1, 18:1, 19:1, or 20:1).

Thus, in an embodiment, the pharmaceutical composition comprises Tween 80 and lecithin at a ratio of from about 5:1 to about 20:1, preferably about 5:1, preferably about 6:1, preferably about 7:1, preferably about 8:1, preferably about 9:1, preferably about 10:1, preferably about 11:1, preferably about 12:1, preferably about 13:1, preferably about 14:1, preferably about 15:1, preferably about 16:1, preferably about 17:1, preferably about 18:1, preferably about 19:1, or more preferably about 20:1.

In a preferred embodiment, the pharmaceutical composition comprises Tween 80 and lecithin at a ratio of about 10:1.

In an embodiment, the pharmaceutical composition comprises Tween 80.

Where the pharmaceutical composition comprises one or more surfactants, the surfactant may be present in a specific ratio with the dispersed oil phase. In certain embodiments, the surfactant to oil ratio is from about 1:10 to about 20:1 (surfactant:oil) (e.g., about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1 (surfactant:oil)).

Thus, in an embodiment, the surfactant to oil ratio is from about 1:10 to about 20:1, preferably about 1:10, preferably about 1:9, preferably about 1:8, preferably about 1:7, preferably about 1:6, preferably about 1:5, preferably about 1:4, preferably about 1:3, preferably about 1:2, preferably about 1:1, preferably about 2:1, preferably about 3:1, preferably about 4:1, preferably about 5:1, preferably about 6:1, preferably about 7:1, preferably about 8:1, preferably about 9:1, preferably about 10:1, preferably about 11:1, preferably about 12:1, preferably about 13:1, preferably about 14:1, preferably about 15:1, preferably about 16:1, preferably about 17:1, preferably about 18:1, preferably about 19:1, or more preferably about 20:1 (surfactant:oil).

In an embodiment, the surfactant to oil ratio is from about 1:1 to about 10:1 (surfactant:oil). In another embodiment, the surfactant to oil ratio is about 5:1. In yet another embodiment, the surfactant to oil ratio is about 1:1.

In an embodiment, the dispersed oil phase comprises the oil and the at least one surfactant at a ratio of from about 1:1 to about 1:10 (oil:surfactant). In another embodiment, the dispersed oil phase comprises the oil and the at least one surfactant at a ratio of about 1:1 (oil:surfactant).

In an embodiment, the dispersed oil phase comprises oleic oil and Tween 80 at a ratio of from about 1:1 to about 1:10 (oil:surfactant). In another embodiment, the dispersed oil phase comprises oleic oil and Tween 80 at a ratio of about 1:1 (oil:surfactant).

In an embodiment, the dispersed oil phase comprises the CBD or a pharmaceutically acceptable salt or functional derivative thereof, the oil and the at least one surfactant at a ratio of about 1:1:1 (CBD:oil:surfactant).

In an embodiment, the dispersed oil phase comprises CBD or a pharmaceutically acceptable salt or functional derivative thereof, oleic oil and Tween 80 at a ratio of about 1:1:1 (CBD:oil:surfactant).

In an embodiment, the dispersed oil phase further comprises one or more or all of the carriers, diluents and excipients selected from the group consisting of polyethylene glycol, propylene glycol and ethanol. In an embodiment, the dispersed oil phase further comprises one or both of the excipients selected from the group consisting of propylene glycol and ethanol.

As used herein, the terms “continuous water phase” and “continuous aqueous phase” are used interchangeably herein to refer to the water in which the dispersed oil phase is distributed. The pharmaceutical composition described herein may therefore be referred to as a “direct emulsion”.

In an embodiment, the pharmaceutical composition is a coarse emulsion.

In another embodiment, the pharmaceutical composition is a nanoemulsion.

Methods for the preparation of emulsions would be known to persons skilled in the art, illustrative examples of which include probe sonication, homogenization and high pressure homogenization. In an embodiment, the emulsions of the present disclosure are prepared by high pressure homogenization.

In an embodiment, the continuous water phase comprises one or more pharmaceutically acceptable carriers, diluents and excipients.

In an embodiment, the continuous water phase comprises one or more pharmaceutically acceptable carriers, diluents and excipients selected from the group consisting of sodium chloride, sodium citrate, citric acid (anhydrous), EDTA, ascorbic acid, sodium metabilsulfite and benzalkonium chloride. In another embodiment, the continuous water phase comprises an excipient selected from the group consisting of sodium metabilsulfite, EDTA, ascorbic acid, sodium chloride, citric acid and combinations of the foregoing. In a preferred embodiment, the continuous water phase comprises an excipient selected from the group consisting of sodium metabilsulfite, EDTA, ascorbic acid, sodium chloride and combinations of the foregoing.

The pharmaceutical compositions described herein may additionally include any suitable additives, carriers, additional therapeutic agents, bioavailability enhancers, side-effect suppressing components, diluents, buffers, flavouring agents, binders, preservatives or other ingredients that are not detrimental to the efficacy of the composition.

In another embodiment, the pharmaceutical composition further comprises one or more additional therapeutic agents. Suitable additional therapeutic agents would be known to persons skilled in the art, illustrative examples of which include anti-anxiety agents, anti-coagulants, anti-convulsants, anti-depressants, muscle relaxants and stimulants.

In an embodiment, the pharmaceutical composition further comprises a volatile anesthetic.

As used herein, the terms “volatile anesthetic” or “VA” refer to a class of anesthetic agents that are liquid at room temperature, but evaporate easily for administration by inhalation. Volatile anesthetics raise the excitatory threshold of neurons and, therefore, may reduce the degree of neuro excitation post-head injury. Suitable volatile anesthetics will be known to persons skilled in the art, illustrative examples of which include methoxyflurane, halothane, enflurane, isoflurane, sevoflurane and desflurane.

In an embodiment, the volatile anesthetic is a halogenated volatile anesthetic, or a pharmaceutically acceptable salt thereof. In another embodiment, the volatile anesthetic is an organofluorine compound. In an embodiment, the organofluorine compound is methoxyflurane or isoflurane. In a preferred embodiment, the organofluorine compound is isoflurane.

Compositions disclosed herein may be prepared according to conventional methods well known in the pharmaceutical and nutraceutical industries, such as those described in Remington's Pharmaceutical Handbook (Mack Publishing Co., NY, USA) using suitable excipients, diluents and fillers. Exemplary additional ingredients include at least one emulsifier (e.g., polyethylene glycol 400 (PEG-400), propylene glycol, vegetable glycol), butylated hydroxytoluene (E321) etc. In general, compositions formulated for administration by inhalation are delivered by MDIs, DPIs, nebulizer solutions, vaporizer solutions, suspensions and nasal sprays (e.g., aqueous and propellant driven).

In an embodiment, the pharmaceutical composition is delivered by nasal spray.

In an embodiment, the pharmaceutic composition is to be delivered by nebulization.

Compositions, e.g., compositions suitable for administration by inhalation, may be presented as discrete units (i.e., dosage forms), each containing a predetermined amount of each component of the composition as an emulsion.

As described elsewhere herein, the compositions may be formulated for administration as separate unit dosage forms for administration. The unit dosage form may be suitable for an inhaler, a nebuliser, an atomiser, an MDI, a nasal spray or a vaporizer.

Effective administration by inhalation requires the production of droplets that can bypass the upper airway and be deposited in the lower respiratory tract. Accordingly, in some embodiments, the nanoscale droplets of the pharmaceutical composition have a droplet size and dispersion that enables penetration into the lower respiratory tract.

In an embodiment, the pharmaceutical composition comprises oil droplets having an average diameter of from about 10 nm to about 500 nm (e.g., 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, or 500 nm).

Thus, in an embodiment, the pharmaceutical composition comprises oil droplets having an average diameter of from about 10 nm to about 500 nm, preferably about 10 nm, preferably about 20 nm, preferably about 30 nm, preferably about 40 nm, preferably about 50 nm, preferably about 60 nm, preferably about 70 nm, preferably about 80 nm, preferably about 90 nm, preferably about 100 nm, preferably about 110 nm, preferably about 120 nm, preferably about 130 nm, preferably about 140 nm, preferably about 150 nm, preferably about 160 nm, preferably about 170 nm, preferably about 180 nm, preferably about 190 nm, preferably about 200 nm, preferably about 210 nm, preferably about 220 nm, preferably about 230 nm, preferably about 240 nm, preferably about 250 nm, preferably about 260 nm, preferably about 270 nm, preferably about 280 nm, preferably about 290 nm, preferably about 300 nm, preferably about 310 nm, preferably about 320 nm, preferably about 330 nm, preferably about 340 nm, preferably about 350 nm, preferably about 360 nm, preferably about 370 nm, preferably about 380 nm, preferably about 390 nm, preferably about 400 nm, preferably about 410 nm, preferably about 420 nm, preferably about 430 nm, preferably about 440 nm, preferably about 450 nm, preferably about 460 nm, preferably about 470 nm, preferably about 480 nm, preferably about 490 nm, or more preferably about 500 nm.

In another embodiment, the pharmaceutical composition comprises oil droplets having an average diameter of from about 100 nm to about 300 nm.

In another embodiment, the pharmaceutical composition comprises oil droplets having an average diameter of about 240 nm.

In an embodiment, the oil droplets have a polydispersity index (PDI) of <0.5 (e.g., 0.5, 0.4, 0.3, 0.2, 0.1, and so on).

In an embodiment, the oil droplets have a PDI of from about 0.2 to about 0.5.

Thus, in an embodiment, the oil droplets have a PDI of from about 0.2 to about 0.5, preferably about 0.2, preferably about 0.3, preferably about 0.4 or more preferably about 0.5. In an embodiment, the oil droplets have a PDI of from about 0.2 to about 0.5, preferably about 0.2, preferably about 0.3, preferably about 0.4 or more preferably about 0.5, as determined by dynamic light scattering (DLS).

Additional physical characteristics, including droplet stability, surface tension, density and viscosity, may also be relevant to the administration of the pharmaceutical compositions described herein.

Droplet stability may be defined by reference to “zeta potential”, which refers to the potential between droplet surface and dispersing liquid medium. Zeta potential is estimated by measuring electrophoretic mobility of the droplets. In certain embodiments, the pharmaceutical composition comprise droplets with a zeta potential ≤−30 mV.

Viscosity is the resistance of the fluid to flow. The specific viscosity of the pharmaceutical compositions described herein may be measured using methods that would be known to persons skilled in the art, illustrative examples of which include the use of capillary tube viscometers. In certain embodiments, the pharmaceutical composition has a viscosity of from about 0 mPA·s to about 3 mPA·s. In another embodiment, the pharmaceutical composition is a low viscosity composition. The term “low viscosity” as used herein refers to compositions with a viscosity of from about 0 mPA·s to about 2 mPA·s.

### Nebulized Pharmaceutical Compositions

In another aspect disclosed herein, there is provided a nebulized pharmaceutical composition comprising an oil-in-water emulsion having a dispersed oil phase and a continuous water phase, wherein the dispersed oil phase comprises CBD or a pharmaceutically acceptable salt or functional derivative thereof.

The term “nebulizer” as used herein refers to a device that aerosolize compositions so that they can be inhaled into the lower respiratory tract.

In an embodiment, the nebulizer is selected from the group consisting of a jet nebulizer, ultrasonic nebulizer, and a vibrating mesh nebulizer.

In a preferred embodiment, the nebulizer is a vibrating mesh nebulizer (e.g., the FOX nebulizer described in Example 3).

In an embodiment, the nebulized pharmaceutical composition comprises oil droplets having an average diameter of from about 10 nm to about 500 nm.

In an embodiment, the oil droplets have an average diameter of ≤300 nm.

In an embodiment, the oil droplets have an average diameter of from about 100 nm to about 300 nm.

In an embodiment, the oil droplets have an average diameter of about 240 nm.

In an embodiment, the oil droplets have a PDI of <0.5.

In an embodiment, the oil droplets have a PDI of ≤0.3.

In an embodiment, wherein the CBD is synthetic CBD.

In an embodiment, the synthetic CBD consists of the (−) CBD enantiomer.

In an embodiment, the nebulized pharmaceutical composition comprises from about 1 mg/mL to about 100 mg/mL CBD or a pharmaceutically acceptable salt or functional derivative thereof.

In an embodiment, the nebulized pharmaceutical composition comprises from about 0.5% (w/v) to about 5% (w/v) CBD or a pharmaceutically acceptable salt or functional derivative thereof. In another embodiment, the nebulized pharmaceutical composition comprises from about 0.5% (w/v) to about 2% (w/v) CBD or a pharmaceutically acceptable salt or functional derivative thereof. In a preferred embodiment, the nebulized pharmaceutical composition comprises about 1% (w/v) CBD or a pharmaceutically acceptable salt or functional derivative thereof.

In an embodiment, the dispersed oil phase comprises an oil selected from the group consisting of olive oil, coconut oil, MCT oil and oleic acid. In a preferred embodiment, the oil is oleic acid.

In an embodiment, the nebulized pharmaceutical composition further comprises at least one surfactant selected from the group consisting of Tween 80, Span 85, DSPC, and lecithin.

In an embodiment, the surfactant is Tween 80.

In an embodiment, the surfactant is Tween 80 and Span 85. In another embodiment, the nebulized pharmaceutical composition comprises Tween 80 and Span 85 at a ratio of from about 1:1 to about 10:1 (Tween 80:Span 85).

In an embodiment, wherein the surfactant is Tween 80 and lecithin. In another embodiment, the nebulized pharmaceutical composition comprises Tween 80 and lecithin at a ratio of from about 5:1 to about 20:1 (Tween 80:lecithin).

In an embodiment, the nebulized pharmaceutical composition further comprises one or more pharmaceutically acceptable carriers, diluents and excipients.

In an embodiment, the pharmaceutically acceptable excipients are selected from the group consisting of polyethylene glycol, ethanol, sodium chloride, sodium citrate, TRIS, citric acid (anhydrous), EDTA, ascorbic acid, sodium metabilsulfite and benzalkonium chloride.

In an embodiment, the nebulized pharmaceutical composition further comprises a volatile anesthetic.

In an embodiment, the volatile anesthetic is an organofluorine compound, or a pharmaceutically acceptable salt thereof. In another embodiment, the organofluorine compound is isoflurane or methoxyflurane. In a preferred embodiment, the organofluorine compound is isoflurane.

In an embodiment, the dispersed oil phase comprises the oil and the at least one surfactant at a ratio of from about 1:1 to about 1:10 (oil:surfactant). In another embodiment, the dispersed oil phase comprises the oil and the at least one surfactant at a ratio of about 1:1 (oil:surfactant).

In an embodiment, the dispersed oil phase comprises the CBD or a pharmaceutically acceptable salt or functional derivative thereof, the oil and the at least one surfactant at a ratio of about 1:1:1 (CBD:oil:surfactant).

In an embodiment, the dispersed oil phase comprises CBD or a pharmaceutically acceptable salt or functional derivative thereof, oleic oil and Tween 80 at a ratio of about 1:1:1 (CBD:oil:surfactant).

In an embodiment, the pharmaceutical composition comprises the formulations in Table 36. In a preferred embodiment, the pharmaceutical composition comprises Formulation 14, Formulation 33, Formulation 35 or Formulation 37 as shown in Table 36.

### Methods for the Treatment of Traumatic Brain Injury (TBI)

The terms “traumatic brain injury” or “TBI” will be understood by persons skilled in the art as meaning an injury to the brain caused by an external force. Common causes include falls, car accidents, assault or being struck by objects during sport. The outcome of a head injury sustained by a subject is determined by two substantially different mechanisms: (i) the primary insult (i.e., primary damage, mechanical damage) occurring at the time of impact; and (ii) the secondary insult (i.e., secondary damage, delayed non-mechanical damage), which represents the consecutive pathological processes initiated at the time of impact with delayed clinical presentation. The primary insult is characterized by direct tissue damage and impaired regulation of cerebral blood flow (CBF) and metabolism. This ischaemia-like pattern leads to accumulation of lactic acid due to anaerobic glycolysis, increased membrane permeability, and consecutive oedema formation. The secondary insult broadly encompasses a range of different pathophysiological outcomes, which may include, for example, terminal membrane depolarisation, releases of excitatory neurotransmitters (e.g., glutamate, aspartate), Ca²⁺- and/or Na²⁺-mediated catabolic intracellular processes, and activation of immunomodulators (e.g., cytokines, prostaglandins, free radicals and complement). Together, these events lead to membrane degradation of vascular and cellular structure and neuronal cell death (e.g., necrotic or apoptotic).

In an embodiment, the pharmaceutical compositions described herein may be useful in reducing or preventing neuronal cell death during the secondary insult of TBI. Accordingly, in an embodiment, the pharmaceutical composition is for use in the treatment or prevention of TBI.

TBI is classified according to its severity: mild, moderate or severe. Classification may be clinically determined based on the Glasgow Coma Scale (GCS) (Ghelichkhani et al., 2018, Emergency (Tehran), 6(1): e42), which assesses motor, verbal and eye-opening responses. A subject with mild TBI will have a GCS of between 13 and 15; a subject with moderate TBI will have a GCS of between 9 and 12; and a subject with severe TBI will have a GCS less than 9. In the absence of a clinical assessment, TBI is considered moderate to severe if there is a loss of consciousness that is longer than 30 minutes and amnesia that lasts for more than 24 hours. If these conditions are not met, TBI is classified as mild. Concussion in the absence of other symptoms is typically classified as a mild TBI.

In an embodiment, the TBI is mild to severe TBI. In another embodiment, the TBI is moderate to severe TBI.

In an embodiment, the subject has acquired TBI while participating in a contact sport.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human. In an embodiment, the subject is an adult. In another embodiment, the subject is a child.

As used herein, the term “effective amount” typically refers to an amount of the pharmaceutical composition described herein that is sufficient to affect one or more beneficial or desired therapeutic outcomes (e.g., reduction in neuronal excitation, reduction in neuronal inflammation, reduction in CBF, reduction in cerebral oxygen consumption, improved recovery from neurocognitive deficit, improved recovery from motor function deficit). Said beneficial or desired therapeutic outcomes may be measured using clinical techniques known in the art, illustrative examples of which include the measurement of cerebral haemoglobin flow (CHbF) and cerebral venous oxyhemoglobin saturation (CS_{VO2}) using near infrared spectroscopy, magnetic resonance imaging (MRI) estimation of global brain oxygen consumption rate as described by Jain et al. (2010, Journal of Cerebral Blood Flow & Metabolism, 30 (9): 1598-1607), quantification of the presence of inflammatory mediators (e.g., Interleukin-1, TNF, TGF-β, etc.), immunohistochemistry for markers of microglial activation (i.e., IBA1), astrocytic response (i.e., GFAP) and neuronal loss (i.e., NeuN or Flurojade), electroencephalography (EEG), and neurocognitive test battery (e.g. ImPACT, Cogstate, etc.) An “effective amount” can be provided in one or more administrations. The exact amount required may vary depending on factors such as the nature and severity of the TBI to be treated, and the age and general health of the subject.

The terms “treat”, “treating”, “treatment” and the like are used interchangeably herein to mean relieving, reducing, alleviating, ameliorating or otherwise inhibiting the severity and/or progression of TBI, or a symptom thereof, in a subject. It is to be understood that the terms “treat”, “treating”, “treatment” and the like, as used herein, do not imply that a subject is treated until clinical symptoms of TBI have been eliminated or are no longer evident (e.g., neuronal excitation, neuronal inflammation, CBF, cerebral oxygen consumption). Said treatment may also reduce the severity of TBI by preventing intracerebral neuronal damage with clinical sequelae.

The terms “prevent”, “preventing”, “prevention” and the like are used interchangeably herein to mean inhibit, hinder, retard, reduce or otherwise delay the development of TBI and/or progression of TBI, or a symptom thereof, in a subject. In the context of the present disclosure, the term “prevent” and variations thereof does not necessarily imply the complete prevention of the specified event. Rather, the prevention may be to an extent, and/or for a time, sufficient to produce the desired effect. Prevention may be inhibition, retardation, reduction or otherwise hindrance of the event, activity or function. Such preventative effects may be in magnitude and/or be temporal in nature.

In an aspect disclosed herein, there is provided a method for the treatment or prevention of TBI comprising administering a therapeutically effective amount of the pharmaceutical composition or nebulized pharmaceutical composition described herein to a subject in need thereof.

In another aspect disclosed herein, there is provided a use of an oil-in-water emulsion having a dispersed oil phase and a continuous water phase in the manufacture of a medicament for the treatment or prevention of TBI, wherein the dispersed oil phase comprises CBD or a pharmaceutically acceptable salt or functional derivative thereof, wherein the medicament is formulated for administration by inhalation.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Example 1—Materials and Methods

Materials used for the preparation of CBD formulation variants are presented in Table 2.

Additional excipients may be included during formulation development that are not listed in Table 2.

### Oil Phase Suitability Screening: CBD Solubility Assessment

To determine the suitability of CBD dissolution in the dispersed phase mixtures (Formulations 1-3), a range of CBD concentrations will be tested. The % w/v prepared for Formulations 1-3 are based on a 10 mL preparation of nanoemulsion. Therefore, to achieve 1-100 mg/mL target dose (1-2 mL volume), 10 mg-1000 mg of CBD will be dissolved.

1-100 mg of CBD powder will be added to each formulation (Formulation 1-3) and visually assessed for compatibility. Subsequent additions of 1-100 mg of CBD powder will be added, with visual assessments taken at each stage to determine the maximum concentration of CBD compatibility with each formulation (i.e., maximum dissolution).

### Nanoemulsion: Proof-of-Concept Trial

Upon reaching maximum dissolution (visually assessed in oil phase suitability screening), a trial of nanoemulsion suitability will be conducted on select formulations. Briefly, dropwise addition of mQ water will be added to the oil phase under magnetic stirring until a total volume of 10 mL is reached. The formulation will then be subjected to a high-energy input method (such as homogenization, probe sonication or high-pressure homogenization).

The selected CBD formulations will be visually assessed. They will also be measured for pH, viscosity, osmolality and DLS as per Table 10.

### Example 2—Exemplary CBD Formulations

Exemplary oil phase CBD formulations are presented in Table 3.

### Preparation—Formulation 1

Weigh 1.5 g (1.425-1.575 g) of propylene glycol and pipette 500 μL (475-525 μL) of EtOH (absolute, 99.8%) into a suitable glassware container. Weigh 500 mg (475-525 mg) of Tween 80 and add to the mixture. Vortex/magnetic stir to allow for complete mixing (if improper mixing persists, bath sonication or another suitable method can be employed).

### Preparation—Formulation 2

Weigh 500 mg (475-525 mg) of oleic acid in a suitable glassware container and add 1.125 g (1.069-1.181 g) of propylene glycol. Pipette 375 μL of EtOH (absolute, 99.8%) and add to the oleic acid mixture. Weigh 500 mg (475-525 mg) of Tween 80 and add to the oleic acid mixture, and vortex/magnetic stir to allow for mixing (if incomplete mixing occurs, bath sonication or another suitable method can be employed).

### Preparation—Formulation 3

Weigh 2 g (1.9-2.1 g) of oleic acid in a suitable glassware container and weigh 500 mg (475-525 mg) of Tween 80 and add to the oleic acid mixture. Vortex/magnetic stir to allow for mixing (if incomplete mixing occurs, bath sonication or another suitable method can be employed).

### Example 3-Preparation of Exemplary CBD Formulations

The production of stable nanoemulsions often requires high-energy input during manufacture. Accordingly, the formulations were manufactured as a coarse emulsion and subjected to three different manufacturing techniques: homogenization (Ultra Turrax), probe sonication (Bandelin Sonoplus HD 3100) and high-pressure homogenization (Emulsiflex C5). The formulations each require a degree of optimization regarding energy setting used and duration of processing (Ultra Turrax and probe sonication) as well as the pressure and number of cycles needed (high-pressure homogenization). Optimization of these parameters was characterized by dynamic light scattering (DLS) particle sizing to determine polydispersity index (PDI) of the formulations. Emulsion stability is largely dictated by particle size and surface charge, therefore criteria for a successful formulation and manufacturing procedure was based on small particle size (e.g., <100-300 nm) with homogenous PDI (e.g., <0.5).

500 mg of CBD was dissolved in each of the oil phase formulations using vortex and bath sonication before being topped to 10 mL with water. Each formulation was then emulsified using probe sonication (Bandelin Sonoplus HD 3100). The emulsions were characterized using DLS, and the results are presented in Table 4.

The formulations were stored at ambient temperature (i.e., 25° C.) and at 5° C. for ˜3 weeks and then assessed visually. Formulation 1 and 3 both developed a yellow tint when stored at ambient temperature and displayed some phase separation when stored at 5° C. Formulation 3 froze at 5° C., likely due to the oleic acid content. Formulation 2 had a slight yellow tint when stored at ambient temperature, but to a lesser extent to that observed for Formulations 1 and 3. No difference in appearance was observed in Formulation 2 when stored at 5° C. in terms of color or phase separation.

Formulation 3 was used to assess the three different manufacturing techniques, as detailed above. The resulting formulations manufactured with each of these methods were assessed using DLS, and the results are presented in Table 5. High-pressure homogenization (Emulsiflex C5) produced the most favorable nanoemulsion properties (i.e., small particle size, low PDI, negative zeta potential).

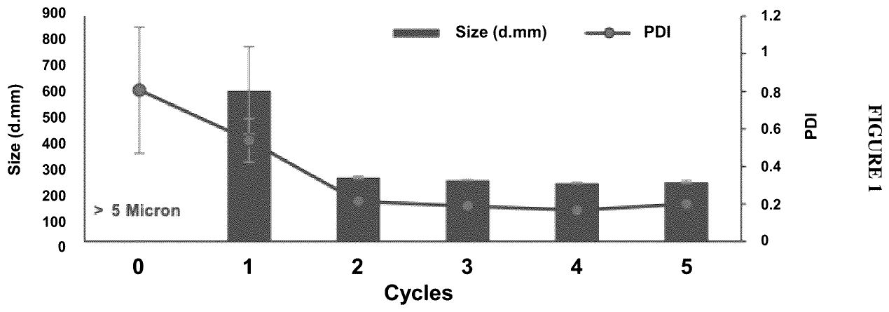

Examination of the average size of the oil droplets after each cycle of high pressure homogenization was conducted to determine the impact of the number of cycles on oil droplet size. As shown in FIG. 1, three cycles of homogenization was sufficient to achieve an average oil droplet diameter of ≤300 nm with a PDI of ≤0.3. Accordingly, high-pressure homogenization, using three cycles with pressure of 15,000-20,000 PSI was selected as a preferred method for the manufacture of the CBD nanoemulsion.

### Example 4-Additional Optimization and Characterization of Emulsion Formulations

Additional optimization of the emulsion formulation will be carried out using an optimized manufacturing process. New excipients may be included such as lipids (e.g., DSPC or soy lecithin), Span 85 and cholesterol. Adjustments of specific concentrations of individual components may be screened-such as co-solvent concentrations (e.g., ethanol and PG), surfactant concentrations and synergistic effects of combined surfactants (e.g., Tween 80 in combination with Span 85).

### Optimization of Surfactant, Oil and CBD Content of Emulsion Formulations

For example, derivatives of Formulation 2 were prepared as shown in Table 6. These additional formulations were developed to assess the effect of varying the ratio of surfactant to oil. The viscosity of Formulations 7 and 8 were measured to be 2.675 and 2.856 mPA, respectively. Formulation 7 was assessed for aerodynamic performance of the formulation and dilutions thereof, initially by DLS to confirm the nanoemulsion properties at the time of manufacture and after 5 days storage. The initial characterization data is presented in Table 7.

A number of selected optimized formulations will be selected for device interaction performance through the FOX® nebulizer system by performing laser diffraction to determine particle size distribution (PSD) and gravimetric determination of output rate.

Dilutions of Formulation 7, i.e., neat, 1:1, 1:3 and 1:7 in water were assessed for aerodynamic performance using the FOX® nebulizer system and the rate of nebulization is presented in Table 8. The 1:7 dilution of Formulation 7 performed the best with an acceptable number of breaths to nebulize 1 mL of the formulation and minimal residual volume in the device.

Furthermore, the addition of a buffer/buffers, preservatives and isotonicity agents will be assessed to determine their suitability with the formulations. Exemplary formulations can be found in Table 9.

As high viscous liquids are known to have poor performance in nebulisers, which was supported by the analysis of dilutions of Formulation 7, and oleic acid is the main contributor to the viscosity of the formulation, two formulations were designed to determine whether the oleic acid content could be reduced. These two formulations are characterised in Table 11.

Addition of water to both formulations led to poor crude emulsion formation with large, visible heterogeneous aggregates forming. These aggregates meant that the formulations were not appropriate for high pressure homogenisation. To determine whether the aggregate formation was due to the high CBD concentration, an additional formulation with the CBD at a 1:1 ratio with the oleic acid and Tween 80 was prepared. Formulation 13 was prepared (Table 12) and characterized by DLS, pH and viscometry (Table 13).

Formulation 13 had acceptable characteristics for a nanoemulsion and the viscosity was low enough to assess the aerodynamic performance of this formulation in a mesh nebulizer (Table 14). These data demonstrate that Formulation 13 was suitable for delivery by mesh nebulizer.

To determine the optimal CBD concentration in a nanoemulsion with a 1:1:1 ratio of CBD:oleic acid:Tween 80 that would perform acceptably in the mesh nebuliser, two formulations were prepared (Table 15). Formulations 14 and 15 were characterized by DLS, pH and viscometry (Table 16). These data demonstrate that Formulation 14 had acceptable particle sizes and PDI values whereas Formulation 15 had larger particles and less homogenous distribution (i.e., higher PDI). Nonetheless, both formulations were assessed for performance in the mesh nebulizer (Table 17). Formulation 14 performed acceptably in the mesh nebulizer. Formulation 15 had a slow output rate and required a large number of breaths. The device also had an early empty detection with Formulation 15 that may be related to foaming of the formulation. In view of these data, Formulation 14 was selected for future formulation development as it contained the most CBD while maintaining acceptable nanoemulsion and aerodynamic characteristics.

To explore whether varying the oleic acid and Tween 80 content in the formulations with 1% CBD would change the performance in the mesh nebulizers, two formulations were prepared (Table 18). Visible particles formed upon addition of water to the oil phase of Formulations 16 and 17. These were much larger in Formulation 16 as compared to Formulation 17. Formulation 17 also was successfully processed by high pressure homogenisation to the milky white appearance of a CBD nanoemulsion. Therefore, Formulation 17 was assessed by DLS, pH and viscometry (Table 19). Compared to Formulation 14, the particle size and PDI for Formulation 17 were larger. However, the values were in an acceptable range so Formulation 17 was further assessed in the mesh nebulizer (Table 20).

To determine the effect of inclusion of a buffer and increased salt concentration on the nebulization characteristics of CBD nanoemulsions, 10 mM phosphate buffered saline (PBS) was used as the aqueous phase (Table 21). Formulation 18 was manufactured using high pressure homogenisation and characterised using DLS, pH and viscometry (Table 22). While the characteristics and aerodynamic performance of Formulation 18 were acceptable (Table 23), visual examination of Formulation 18 after ˜1 week revealed that the addition of PBS resulted in poor stability of the nanoemulsion over time.

Based on the results obtained that indicate the optimal oil and surfactant content for the CBD nanoemulsion was about 1%, Formulation 1 was adjusted to remove oil and assess the performance of probe sonication rather than high pressure homogenization (table 24). To understand whether there was any benefit to oleic acid removal this formulation was also downsize using probe sonication. Formulations 20 and 21 were characterized using DLS, pH and viscometry (Table 25).

Because of the poor sample quality of Formulation 20 (e.g., large particle size and PDI along with formation of particle sediment) it was not characterised for pH, viscosity or in the mesh nebuliser. However, Formulation 21 was characterized in the mesh nebuliser (Table 26). There was some early empty detection in the device due to foaming, however, this occurred after >95% of the dose had been delivered.

Taken together, these data demonstrate that the distributed oil phase performance improves where the ratio of CBD:oil:surfactant is 1:1:1. Optimal performance of the CBD nanoemulsion in the mesh nebulizer was observed where the concentration of CBD, oil and surfactant in the nanoemulsion was 1%, respectively.

Accordingly, preferred CBD nanoemulsion formulations comprise 1% oleic acid, 1% Tween 80, 1.41% propylene glycol, 0.47% ethanol and 1% CBD using water as the aqueous phase (i.e., Formulation 14). As shown herein, the use of PBS as the aqueous phase impacted the stability of the CBD nanoemulsion.

### PSD by Laser Diffraction

PSD determinations will be performed at 15 L/min using a different FOX® head per formulation and one FOX® base unit using an appropriate fill volume.

Particle volume (VMD), grain size distribution (GSD) and fine particle fraction (FPF)≤5 μm and refractive index will be measured as appropriate.

### Gravimetric Delivered Dose Determinations

Delivered dose (DD) determinations will be performed at 15 L/min using a different FOX® head per formulation using an appropriate fill volume. The DD and continuous output rate (COR) will be determined on each formulation using the mass change from full to empty as a surrogate for delivered dose.

### Example 5-Stability Assessment

Batches of selected formulations will be manufactured and analyzed for stability over a 6-month period. Each batch will be incubated at 5° C., 25° C./60% RH and 40° C./75% RH. Aliquots will be removed at 1, 2, 3 and 6 months and assessed for appearance, CBD content/breakdown products, pH and impurities. At the 3 and 6 month time points the formulations will also be assessed for osmolality, viscosity, nebulized particle size distribution and continuous output and delivered dose.

When Formulations 1-21 were stored for more than 2 days they started to develop a peach or yellow colour. This was thought to be oxidation of one or more components of the formulation. To reduce oxidation and improve the stability, formulations with the addition of antioxidants were prepared (Table 27). The antioxidant chemicals and concentrations thereof are all on the FDA list of permitted ingredients for drugs delivered to the lungs. Antioxidants were dissolved in water and the pH adjusted to 6.5-8.5 with NaOH. All of the formulations were downsized using high pressure homogenisation. Each of these formulations was initially assessed for appearance, pH, CBD content and related substances using a non-validated HPLC method, viscosity, conductivity, DLS (Table 28), before being characterized for performance in the mesh nebulizer (Table 29).

Once Formulations 31-37 were confirmed to have acceptable solution characteristics and performance in the mesh nebulizer, aliquots were taken and stored with refrigeration, at 5° C. Aliquots were removed at 1, 2, 4 and 6 weeks as well as 2 and 3 months and analyzed for appearance, CBD content and related substances using a non-validated HPLC method and DLS (Tables 30-35). Formulation 31, which included ascorbic acid, had a decrease in pH at week 6 and an gradual increase in average size reaching 301 nm by month 3. This was the only formulation to have an average particle size of >300 nm at any point during the study. Formulations 31, 34 and 36 all had greater than 5% reduction in CBD content as assessed by HPLC at month 3, which is out of the acceptance criteria in the ICH Q1A (R2) guidelines. Formulations 32, 34 and 36 were all noticeably yellow by month 3 and Formulation 31 had a slight yellow colouration.

Formulations 33, 35 and 37 all retained the target product profile for all DLS parameters and CBD content and did not turn yellow with 3 months storage at 5° C.

Taken together, these data demonstrate that the addition of sodium metabisulfite, EDTA, ascorbic acid and sodium chloride improve the stability of CBD nanoemulsions during storage at 5° C. for 3 months.

### DISCUSSION

Collectively, these data are enabling for the preparation of CBD formulations for administration by inhalation. In particular, it has been shown that nebulized CBD formulations can be prepared utilizing oils/lipids along with specific surfactants and co-solvents to generate oil in water emulsions to achieve adequate dissolution of the molecule . . . . The lipophilic active pharmaceutical ingredient (API; i.e., CBD) of the pharmaceutical compositions described herein can added to the oil phase (i.e., dispersed phase) of the formulation, before addition to the continuous phase (water) and under high-energy input, can generate nanoscale droplets of oil containing CBD suspended throughout water.

TABLE 1
Cannabidiol and related cannabinoids

| | | Chemical |
|---|---|---|
| | | properties/ |
| | | [M + H]⁺ ESI |
| Name | Structure | MS |
| cannabidiol (CBD) | 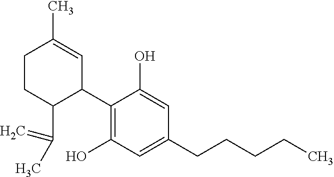 [SMILES: C=C(C)C1CCC(C)=CC1c1c(O)cc(CCCCC)cc1O] | decarboxylation product of CBDA m/z 315.2319 |
| cannabidiolic acid (CBDA) | 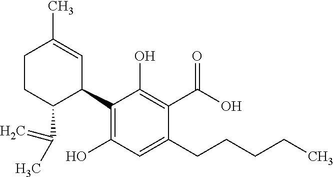 [SMILES: C=C(C)[C@@H]1CCC(C)=C[C@H]1c1c(O)cc(CCCCC)c(C(=O)O)c1O] | m/z 359.2217 |
| cannabigerolic acid (CBGA) | 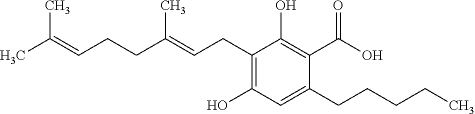 [SMILES: CCCCCc1cc(O)c(C/C=C(\C)CCC=C(C)C)c(O)c1C(=O)O] | m/z 361.2373 |

TABLE 2
List of materials

| Material | Molecular weight (g/mol) |
|---|---|
| Cannabidiol (CBD) | 314.5 |
| Oleic acid | 282.47 |
| Polysorbate 80 (PS80/Tween 80) | 1310 |
| Sorbitan trioleate (Span 85) | 957.5 |
| mQ water | 18.02 |
| Sodium chloride (NaCl) | 294.1 |
| Propylene glycol (PG; 1,2-propanediol) | 76.09 |
| Ethanol | 46.07 |
| Soy lecithin | 643.9 |
| DSPC | 734.039 |
| Cholesterol | 386.65 |
| EDTA | 292.2438 |
| Citric acid | 192.124 |
| Tri-sodium citrate dihydrate | 294.1 |
| Benzalkonium chloride | 372.028 |

TABLE 3
Exemplary oil phase formulations comprising 50-
100 mg (minimum) CBD powder in a 10 mL volume

| | Excipients | | Oil | Surfactant | |
|---|---|---|---|---|---|
| | PG | EtOH | Oleic acid | Tween80 | |
| Formulation | (%, w/v) | (%, v/v) | (%, w/v) | (%, w/v) | Comment |
| 1 | 15 | 5 | 0 | 5 | Oleic acid |
| | | | | | free |
| 2 | 11.25 | 3.75 | 5 | 5 | Combination |
| | | | | | of oil and |
| | | | | | co-solvent |
| 3 | 0 | 0 | 20 | 5 | Co-solvent |
| | | | | | free |

TABLE 4
Characterization of Formulation 1, 2 and 3 by DLS

| | Average droplet | | Average zeta |
|---|---|---|---|
| Formulation | Size (nm) | Average PDI | potential (mV) |
| 1 | 721.7 | 0.631 | −22.1 |
| 2 | 265.8 | 0.310 | −35.1 |
| 3 | 388.4 | 0.256 | −35.3 |

TABLE 5
Characterization of Formulation 3 manufactured
using different methods

| | | Average | | |
|---|---|---|---|---|
| | Manufacturing | droplet Size | Average | Average zeta |
| Formulation | method | (nm) | PDI | potential (mV) |
| 4 | Probe sonication | 356.2 | 0.24 | −39.0 |
| 5 | Homogenization | 3171.1 | 1.0 | −36.1 |
| 6 | High-pressure | 237.5 | 0.2 | −31.9 |
| | homogenization | | | |

TABLE 6
Formulations for assessment of surfactant to oil ratio

| | Excipients | | Oil | Surfactant | |
|---|---|---|---|---|---|
| Formu- | PG | EtOH | Oleic acid | Tween 80 | |
| lation | (%, w/v) | (%, w/v) | (%, w/v) | (%, w/v) | Comment |
| 2 | 11.25 | 3.75 | 5 | 5 | 1:1 |
| | | | | | (surfactant:oil) |
| 7 | 11.25 | 3.75 | 1.67 | 8.33 | 5:1 |
| | | | | | (surfactant:oil) |
| 8 | 11.25 | 3.75 | 0.91 | 9.09 | 10:1 |
| | | | | | (surfactant:oil) |

TABLE 7
Characterization of Formulation 7 and 8 by DLS

| | | Average | | Average zeta | Average |
|---|---|---|---|---|---|
| | Time | droplet Size | Average | potential | viscosity |
| Formulation | (day) | (nm) | PDI | (mV) | (mPA*s) |
| 7 | 0 | 244.73 | 0.33 | −37.9 | 2.67 |
| | 5 | 239.93 | 10.36 | — | |
| 8 | 0 | 242.20 | 0.298 | −34.5 | 2.86 |

TABLE 8
Performance of Formulation 7 and dilutions
thereof in a mesh nebulizer

| | | | | Residual | |
|---|---|---|---|---|---|
| | CBD (%, | Number of | Delivered | volume | Output rate |
| Dilution | w/v) | breaths | dose (g) | (mL) | (OR; g/min) |
| Neat | 5 | ND | 0.174 | 72.1 | ND |
| 1:1 | 2.5 | 300 | 0.9529 | 33.7 | 0.06 |
| 1:3 | 1.25 | 154 | 1.0015 | 0.5 | 0.13 |
| 1:7 | 0.625 | 76 | 0.9953 | 1.4 | 0.26 |

TABLE 9
Formulation adjustments for optimization

| Formulation | Optimisation Parameter | Comment |
|---|---|---|
| 1-3 | Oil and surfactant | Adjustments in the oleic acid and Tween 80 |
| | phase | concentrations can be done to achieve |
| | | improved API loading and smaller particle |
| | | size/PDI. Generally, increasing surfactant |
| | | concentration can reduce particle size. |
| | | Furthermore, additional excipients can be |
| | | added if required, such as soy lecithin and |
| | | Span 85, particularly with a synergistic |
| | | approach to a combination of surfactants |
| | | (e.g., Tween 80: Span 85 75:25 ratio). |
| 4-6 | Co-solvents | Adjustments to co-solvent concentrations |
| | | (such as PG and EtOH) can be done. |
| 7-9 | Buffer/isotonicity | A suitable buffer and pH will be selected and |
| | | screened for nanoemulsion compatibility. |
| | | NaCl can be added to adjust isotonicity of |
| | | the formulation. Assess nanoemulsion |
| | | compatibility. Examples include Sodium |
| | | Citrate, TRIS and Citric Acid (anhydrous). |
| 10-12 | Preservatives | FDA approved preservatives can be added to |
| | | improve stability. Compatibility of the |
| | | formulation and the preservative will be |
| | | determined. Examples include EDTA, |
| | | ascorbic acid and benzalkonium chloride. |
| Example | Oleic Acid (x % w/v), PG | Comparison of synergistic co-surfactants |
| formulation | (15% w/v), Ethanol (5% | (Tween 80 and Span 85 oleic acid |
| 1 | v/v), Span 85 (2.5% | concentration determined by previous |
| | w/v), Tween 80 (2.5% | screening. |
| | w/v) and mQ Water | |
| | (topped to 10 mL). | |
| Example | Oleic Acid (x % w/v), PG | Inclusion of lipid (lecithin) as a co-surfactant |
| formulation | (15% w/v), Ethanol (5% | alongside Tween 80. |
| 2 | v/v), Tween 80 (1% w/v), | |
| | Lecithin (0.1% w/v) and | |
| | mQ Water (topped to 10 | |
| | mL). | |

TABLE 10
Testing plan for CBD formulations

| Test | Replicate (n) |
|---|---|
| Visual assessment | 1 |
| pH | 1 |
| Viscosity | 3 |
| Osmolality | 3 |
| Emulsion stability | 1 |
| Particle size distribution (PSD) | 3 (appropriate fill volume) |
| Gravimetric delivered dose (DD) and | 3 (appropriate fill volume) |
| continued output rate (COR) determination | |

TABLE 11
Formulations to assess the effect of reducing the oleic acid content

| Formu- | OA | T80 | PG | EtOH | CBD | |
|---|---|---|---|---|---|---|
| lation | (%, w/v) | (%, w/v) | (%) | (%) | (%, w/v) | Comments |
| 9 | 0.63 | 0.63 | 1.41 | 0.47 | 3 | Oleic acid |
| | | | | | | reduction |
| 10 | 0.63 | 0.63 | 0 | 1.88 | 3 | Oleic acid |
| | | | | | | reduction |
| | | | | | | and PG |
| | | | | | | replace |
| | | | | | | with EtOH |

TABLE 12
Formulations to assess the effect of high
CBD concentration on aggregate formation

| | OA | T80 | | | CBD | |
|---|---|---|---|---|---|---|
| Formu- | (%, | (%, | PG | EtOH | (%, | |
| lation | w/v) | w/v) | (%) | (%) | w/v) | Comments |
| 13 | 0.63 | 0.63 | 1.41 | 0.47 | 0.63 | 1:1:1 ratio |
| | | | | | | CBD:oleic |
| | | | | | | acid:Tween80 |

TABLE 13
Characterization of Formulation 13 by DLS

| | Average | | Average zeta | | Average |
|---|---|---|---|---|---|
| | droplet Size | | potential | | viscosity |
| Formulation | (nm) | Average PDI | (mV) | pH | (mPA*s) |
| 13 | 237.6 ± 4.6 | 0.307 ± 0.9 | −36.1 ± 0.9 | 6.9 | 1.0 ± 0.0 |

TABLE 14
Performance of Formulation 13 in a mesh nebulizer

| | | Delivered dose | Number of | Output rate |
|---|---|---|---|---|
| Formulation | CBD (%, w/v) | (%, nominal) | breaths | (g/min) |
| 13 | 0.63 | 92.8 ± 3.8 | 65 ± 17.6 | 0.30 ± 0.1 |

TABLE 15
Formulations to assess upper limits
of CBD concentration for nebulization

| | OA (%, | T80 (%, | | | CBD (%, |
|---|---|---|---|---|---|
| Formulation | w/v) | w/v) | PG (%) | EtOH (%) | w/v) |
| 14 | 1 | 1 | 1.41 | 0.47 | 1 |
| 15 | 1.5 | 1.5 | 1.41 | 0.47 | 1.5 |

TABLE 16
Characterization of Formulations 14 and 15 by DLS

| | Average | | Average zeta | | Average |
|---|---|---|---|---|---|
| | droplet Size | | potential | | viscosity |
| Formulation | (nm) | Average PDI | (mV) | pH | (mPA*s) |
| 14 | 236.1 ± 10.4 | 0.273 ± 0.03 | −45.8 ± 2.1 | 6.7 | 1.04 ± 0.0 |
| 15 | 261.6 ± 20.0 | 0.322 ± 0.02 | −41.7 ± 0.4 | 6.2 | 1.08 ± 0.0 |

TABLE 17
Performance of Formulations 14 and 15 in a mesh nebulizer

| | CBD | Delivered dose | Number of | Output rate |
|---|---|---|---|---|
| Formulation | (%, w/v) | (%, nominal) | breaths | (g/min) |
| 14 | 1 | 99.5 ± 2.6 | 91 ± 7.2 | 0.22 ± 0.0 |
| 15 | 1.5 | 98.5 ± 2.7 | 183 ± 13.0 | 0.11 ± 0.0 |

TABLE 18
Formulations to assess the effect of varying
oil and surfactant content with 1% CBD

| | Oleic | Tween | Propylene | | | |
|---|---|---|---|---|---|---|
| Formu- | acid | 80 | glycol | Ethanol | CBD | |
| lation | (% w/v) | (% w/v) | (%) | (%) | (% w/v) | Comments |
| 16 | 1 | 0.63 | 1.41 | 0.47 | 1 | Reduced |
| | | | | | | tween 80 |
| 17 | 0.63 | 1 | 1.41 | 0.47 | 1 | Reduced |
| | | | | | | Oleic acid |

TABLE 19
Characterization of Formulation 17

| | | | Zeta | | |
|---|---|---|---|---|---|
| | Size | | potential | | |
| Formu- | average | PDI | average | | Viscosity |
| lation | (nm) | average | (mV) | pH | (mPA*s) |
| 17 | 321.7 ± 119.6 | 0.339 ± 0.02 | −40.4 ± 1.6 | 6.8 | 1.03 ± 0.0 |

TABLE 20
Performance of Formulation 17 in a mesh nebulizer

| | | Delivered dose | Number of | Output rate |
|---|---|---|---|---|
| Formulation | CBD (% w/v) | (% nominal) | breaths | (g/min) |
| 17 | 1 | 100.9 | 106 | 0.19 |

TABLE 21
Formulation to assess the effect of using PBS as the aqueous phase

| | Oleic | Tween | Propylene | | | |
|---|---|---|---|---|---|---|
| Formu- | acid | 80 | glycol | Ethanol | CBD | |
| lation | (% w/v) | (% w/v) | (%) | (%) | (% w/v) | Comments |
| 18 | 1 | 1 | 1.41 | 0.47 | 1 | PBS used |
| | | | | | | for |
| | | | | | | aqueous |
| | | | | | | phase in |
| | | | | | | place of |
| | | | | | | water |

TABLE 22
Characterization of Formulation 18

| | | | Zeta | | |
|---|---|---|---|---|---|
| | | | potential | | |
| | Size average | | average | | Viscosity |
| Formulation | (nm) | PDI average | (mV) | pH | (mPA*s) |
| 18 | 222.6 ± 1.3 | 0.241 ± 0.01 | −70.3 ± 3.8 | 6.8 | 1.03 ± 0.0 |

TABLE 23
Performance of Formulation 18 in a mesh nebulizer

| | | Delivered dose | Number of | Output rate |
|---|---|---|---|---|
| Formulation | CBD (% w/v) | (% nominal) | breaths | (g/min) |
| 18 | 1 | 97.6 | 78 ± 18.8 | 0.26 ± 0.1 |

TABLE 24
Formulations to re-assess oil removal and
probe sonication with the 1% oil phase

| | Oleic | Tween | | | | |
|---|---|---|---|---|---|---|
| | acid | 80 | Propylene | | CBD | |
| Formu- | (% | (% | glycol | Ethanol | (% | |
| lation | w/v) | w/v) | (%) | (%) | w/v) | Comments |
| 20 | 0 | 1 | 3 | 1 | 1 | Re-testing |
| | | | | | | oleic acid |
| | | | | | | removal. |
| | | | | | | Need to be |
| | | | | | | downsized |
| | | | | | | by probe |
| | | | | | | sonication |
| | | | | | | due to |
| | | | | | | particle |
| | | | | | | size after |
| | | | | | | addition of |
| | | | | | | water. |
| 21 | 1 | 1 | 1 | .47 | 1 | Downsized |
| | | | | | | using probe |
| | | | | | | sonication in |
| | | | | | | place of high |
| | | | | | | pressure |
| | | | | | | homogenisation |

TABLE 25
Characterization of Formulations 20 and 21

| | | | Zeta | | |
|---|---|---|---|---|---|
| | | | potential | | |
| | Size average | | average | | Viscosity |
| Formulation | (nm) | PDI average | (mV) | pH | (mPA*s) |
| 20 | 373.2 ± 34.6 | 0.383 ± 0.04 | −44.1 ± 1.1 | nd | nd |
| 21 | 187.7 ± 2.5 | 0.245 ± 0.01 | −49.4 ± 2.4 | 6.3 | 1.06 ± 0.0 |

TABLE 26
Performance of Formulation 21 in a mesh nebulizer

| | | Delivered dose | Number of | Output rate |
|---|---|---|---|---|
| Formulation | CBD (% w/v) | (% nominal) | breaths | (g/min) |
| 21 | 1 | 98.3 ± 0.5 | 84 ± 31.9 | 0.26 ± 0.1 |

TABLE 27
Formulations for assessing the oxidation
and stability of a CBD nanoemulsion

| | Oleic | Tween | | | | |
|---|---|---|---|---|---|---|
| | acid | 80 | Propylene | | CBD | |
| Formu- | (% | (% | glycol | Ethanol | (% | |
| lation | w/v) | w/v) | (%) | (%) | w/v) | Antioxidant |
| 31 | 1 | 1 | 1.41 | 0.47 | 1 | 2.3 mM |
| | | | | | | ascorbic acid |
| 32 | 1 | 1 | 1.41 | 0.47 | 1 | 20 mM |
| | | | | | | citric acid |
| | | | | | | (anhyrdrous) |
| 33 | 1 | 1 | 1.41 | 0.47 | 1 | 15.8 mM |
| | | | | | | sodium |
| | | | | | | metabisulfite |
| 34 | 1 | 1 | 1.41 | 0.47 | 1 | Control |
| 35 | 1 | 1 | 1.41 | 0.47 | 1 | 1.7 mM EDTA |
| 36 | 1 | 1 | 1.41 | 0.47 | 1 | Bath |
| | | | | | | sonication |
| | | | | | | degassing |
| 37 | 1 | 1 | 1.41 | 0.47 | 1 | 2.3 mM |
| | | | | | | ascorbic acid |
| | | | | | | and 154 mM |
| | | | | | | sodium |
| | | | | | | chloride |

TABLE 28
Characterization of Formulations 31-37

| | | | Zeta | | | | |
|---|---|---|---|---|---|---|---|
| | Size | | potential | | | | |
| | average | PDI | average | | viscosity | Conductivity | CBD |
| Formulation | (nm) | average | (mV) | pH | (mPA*s) | (μS/cm) | (mg/mL) |
| 31 | 226.5 ± | 0.28 ± | −50.7 ± | 6.7 | 1.02 ± | 163.4 | 9.82 |
| | 1.0 | 0.0 | 0.6 | | 0.01 | | |
| 32 | 224.4 ± | 0.36 ± | −76.6 ± | 6.8 | 1.03 ± | 3523.5 | 9.22 |
| | 4.8 | 0.02 | 1.4 | | 0.00 | | |
| 33 | 208.4 ± | 0.29 ± | −69.7 ± | 6.4 | 1.02 ± | 2950.7 | 9.62 |
| | 2.5 | 0.02 | 1.7 | | 0.00 | | |
| 34 | 224.4 ± | 0.33 ± | −47.2 ± | 6.5 | 1.03 ± | 22.8 | 10.10 |
| | 2.8 | 0.04 | 1.1 | | 0.00 | | |
| 35 | 222.7 ± | 0.32 ± | −64.3 ± | 6.9 | 1.02 ± | 497.3 | 9.86 |
| | 0.8 | 0.02 | 0.5 | | 0.01 | | |
| 36 | 224.5 ± | 0.34 ± | −47.4 ± | 6.9 | 1.00 ± | 37.1 | 9.46 |
| | 1.9 | 0.01 | 0.5 | | 0.00 | | |
| 37 | 292.3 ± | 0.37 + | −53.4 + | 5.5 | 1.03 ± | 12835.7 | 9.42 |
| | 8.4 | 0.02 | 1.1 | | 0.00 | | |

TABLE 29
Performance of Formulations 31-37 in a mesh nebulizer

| | | Delivered dose | Number of | Output rate |
|---|---|---|---|---|
| Formulation | CBD (% w/v) | (% nominal) | breaths | (g/min) |
| 31 | 1 | 98.8 ± 0.2 | 100 ± 22 | 0.21 ± 0.05 |
| 32 | 1 | 97.9 ± 4.1 | 101 ± 21 | 0.20 ± 0.03 |
| 33 | 1 | 99.8 ± 1.4 | 99 ± 18 | 0.21 ± 0.04 |
| 34 | 1 | 97.3 ± 1.1 | 94 ± 24 | 0.22 ± 0.06 |
| 35 | 1 | 98.5 ± 1.6 | 101 ± 21 | 0.20 ± 0.04 |
| 36 | 1 | 99.1 ± 1.5 | 108 ± 19 | 0.19 ± 0.03 |
| 37 | 1 | 99.0 ± 0.2 | 119 ± 23 | 0.17 ± 0.03 |

TABLE 30
Assessment of pH of CBD nanoemulsions stored at 5° C. over time

| Formulation | T = 0 | 1 week | 2 week | 4 week | 6 week | 2 month | 3 month | Comment |
|---|---|---|---|---|---|---|---|---|
| 31 | 6.7 | 6.4 | 6.4 | 6.4 | 5.7 | 5.6 | 5.6 | Decrease |
| | | | | | | | | at 6 week |
| 32 | 6.8 | 6.8 | 6.8 | 6.9 | 6.8 | 6.8 | 6.8 | steady |
| 33 | 6.4 | 6.3 | 6.2 | 6.2 | 5.9 | 5.9 | 5.4 | Slow |
| | | | | | | | | decrease |
| | | | | | | | | over time |
| 34 | 6.5 | 6.7 | 6.7 | 6.7 | 6.4 | 6.6 | 6.4 | fluctuates |
| 35 | 6.9 | 6.7 | 6.6 | 6.7 | 6.5 | 6.5 | 6.4 | Slow |
| | | | | | | | | decrease |
| | | | | | | | | over time |
| 36 | 6.9 | 6.7 | 6.8 | 6.7 | 6.4 | 6.6 | 6.4 | Slow |
| | | | | | | | | decrease |
| | | | | | | | | over time |
| 37 | 5.5 | 5.6 | 5.6 | 5.6 | 5.4 | 5.4 | 5.1 | Slow |
| | | | | | | | | decrease |
| | | | | | | | | over time |

TABLE 31
Assessment of average particle size of CBD
nanoemulsions stored at 5° C. over time

| Formulation | T = 0 | 1 week | 2 week | 4 week | 6 week | 2 month | 3 month |
|---|---|---|---|---|---|---|---|
| 31 | 226.5 ± | 243.8 ± | 219.6 ± | 228.2 ± | 229.6 ± | 246.3 ± | 302.1 ± |
| | 1.0 | 0.8 | 1.8 | 1.6 | 1.7 | 1.8 | 2.1 |
| 32 | 222.4 ± | 254.2 ± | 219.1 ± | 218.4 ± | 212.1 ± | 251.9 ± | 220.6 ± |
| | 4.8 | 4.0 | 4.9 | 4.4 | 0.7 | 5.7 | 1.7 |
| 33 | 208.4 ± | 206.0 ± | 209.0 ± | 210.7 ± | 210.3 ± | 208.8 ± | 209.8 ± |
| | 2.5 | 2.2 | 1.5 | 0.4 | 3.7 | 1.9 | 3.0 |
| 34 | 224.4 ± | 222.4 ± | 223.1 ± | 250.3 ± | 225.3 ± | 228.1 ± | 225.3 ± |
| | 2.8 | 2.7 | 0.6 | 2.6 | 4.6 | 1.9 | 2.1 |
| 35 | 222.7 ± | 216.4 ± | 212.3 ± | 214.9 ± | 213.8 ± | 321.1 ± | 216.3 ± |
| | 0.8 | 3.4 | 1.8 | 2.0 | 3.3 | 6.0 | 2.5 |
| 36 | 224.5 ± | 226.2 ± | 220.8 ± | 218.3 ± | 226.9 ± | 274.6 ± | 280.8 ± |
| | 1.9 | 5.4 | 1.0 | 2.7 | 4.1 | 7.2 | 14.9 |
| 37 | 292.3 ± | 228.6 ± | 226.9 ± | 229.3 ± | 228.8 ± | 285.3 ± | 225.6 ± |
| | 8.4 | 2.0 | 3.7 | 4.9 | 2.9 | 19.9 | 2.8 |

TABLE 32
Assessment of PDI of CBD nanoemulsions stored at 5° C. over time

| Formulation | T = 0 | 1 week | 2 week | 4 week | 6 week | 2 month | 3 month |
|---|---|---|---|---|---|---|---|
| 31 | 0.28 ± | 0.38 ± | 0.30 ± | 0.34 ± | 0.33 ± | 0.35 ± | 0.37 ± |
| | 0.00 | 0.03 | 0.02 | 0.02 | 0.01 | 0.01 | 0.03 |
| 32 | 0.36 ± | 0.41 ± | 0.34 ± | 0.29 ± | 0.30 ± | 0.34 ± | 0.34 ± |
| | 0.02 | 0.04 | 0.02 | 0.01 | 0.02 | 0.02 | 0.01 |
| 33 | 0.29 ± | 0.28 ± | 0.25 ± | 0.29 ± | 0.26 ± | 0.28 ± | 0.30 ± |
| | 0.02 | 0.02 | 0.01 | 0.02 | 0.01 | 0.01 | 0.01 |
| 34 | 0.33 ± | 0.31 ± | 0.31 ± | 0.33 ± | 0.31 ± | 0.33 ± | 0.30 ± |
| | 0.04 | 0.03 | 0.02 | 0.05 | 0.04 | 0.02 | 0.04 |
| 35 | 0.32 ± | 0.26 ± | 0.28 ± | 0.28 ± | 0.28 ± | 0.52 ± | 0.28 ± |
| | 0.02 | 0.01 | 0.00 | 0.01 | 0.02 | 0.10 | 0.02 |
| 36 | 0.34 ± | 0.34 ± | 0.33 ± | 0.31 ± | 0.33 ± | 0.36 ± | 0.36 ± |
| | 0.01 | 0.01 | 0.04 | 0.03 | 0.03 | 0.01 | 0.02 |
| 37 | 0.37 ± | 0.37 ± | 0.34 ± | 0.36 ± | 0.35 ± | 0.39 ± | 0.32 ± |
| | 0.02 | 0.01 | 0.04 | 0.01 | 0.02 | 0.02 | 0.02 |

TABLE 33
Assessment of zeta potential of CBD nanoemulsions stored at 5° C. over time

| Formulation | T = 0 | 1 week | 2 week | 4 week | 6 week | 2 month | 3 month |
|---|---|---|---|---|---|---|---|
| 31 | −50.7 ± | −47.2 ± | −50.3 ± | −48.6 ± | −61.0 ± | −57.5 ± | −64.8 ± |
| | 0.6 | 0.7 | 0.9 | 1.4 | 0.9 | 1.6 | 2.5 |
| 32 | −76.6 ± | −77.6 ± | −74.0 ± | −78.1 ± | −75.8 ± | −77.6 ± | −78.2 ± |
| | 1.4 | 0.8 | 0.8 | 1.4 | 2.6 | 1.7 | 0.9 |
| 33 | −69.7 ± | −69.6 ± | −66.1 ± | −68.3 ± | −68.0 ± | −71.2 ± | −66.2 ± |
| | 1.7 | 0.5 | 2.0 | 0.7 | 1.5 | 1.5 | 2.3 |
| 34 | −47.2 ± | −47.8 ± | −46.9 ± | −48.1 ± | −56.9 ± | −52.2 ± | −62.7 ± |
| | 1.1 | 0.2 | 1.5 | 1.0 | 4.0 | 1.1 | 1.8 |
| 35 | −64.3 ± | −63.6 ± | −57.6 ± | −62.7 ± | −67.8 ± | −63.2 ± | −74.0 ± |
| | 0.5 | 0.9 | 2.2 | 0.4 | 2.6 | 1.2 | 2.2 |
| 36 | −47.4 ± | −45.5 ± | −46.1 ± | −67.4 ± | −61.5 ± | −50.4 ± | −59.0 ± |
| | 0.5 | 0.9 | 0.2 | 1.2 | 0.8 | 1.8 | 0.7 |
| 37 | −53.4 ± | −51.6 ± | −42.5 ± | −61.6 ± | −60.5 ± | −48.5 ± | −67.9 ± |
| | 1.1 | 0.2 | 2.7 | 1.7 | 0.6 | 1.3 | 1.2 |

TABLE 34
Assessment of CBD concentration in mg/mL by HPLC
of CBD nanoemulsions stored at 5° C. over time

| Formu- | | | | | | | |
|---|---|---|---|---|---|---|---|
| lation | T = 0 | 1 week | 2 week | 4 week | 6 week | 2 month | 3 month |
| 31 | 9.82 | 9.87 | 9.78 | 9.91 | 9.53 | 9.65 | 9.27 |
| 32 | 9.22 | 9.35 | 9.16 | 9.19 | 9.13 | 9.19 | 8.85 |
| 33 | 9.62 | 9.87 | 9.64 | 9.63 | 9.52 | 9.51 | 9.32 |
| 34 | 10.1 | 10.04 | 9.84 | 9.81 | 9.57 | 9.54 | 9.09 |
| 35 | 9.86 | 10.09 | 9.9 | 9.93 | 9.85 | 9.84 | 9.64 |
| 36 | 9.46 | 9.54 | 9.24 | 9.39 | 8.95 | 9.14 | 8.67 |
| 37 | 9.42 | 9.62 | 9.41 | 9.31 | 9.31 | 9.43 | 9.21 |

TABLE 35
Change in CBD concentration expressed as percent relative to T =
0 for CBD nanoemulsions stored at 5° C. over time

| Formu- | | | | | | | |
|---|---|---|---|---|---|---|---|
| lation | T=0 | 1 week | 2 week | 4 week | 6 week | 2 month | 3 month |
| 31* | — | 0.5 | −0.5 | 0.9 | −3 | −1.8 | −5.6 |
| 32 | — | 1.4 | −0.6 | −0.3 | −1 | −0.4 | −4 |
| 33 | — | 2.6 | 0.2 | 0.1 | −1 | −1.2 | −3.1 |
| 34* | — | −0.6 | −2.6 | −2.8 | −5.2 | −5.6 | −10 |
| 35 | — | 2.3 | 0.4 | 0.7 | −0.2 | −0.2 | −2.3 |
| 36* | — | 0.8 | −2.3 | −0.7 | −5.4 | −3.4 | −8.4 |
| 37 | — | 2.1 | −0.1 | −1.2 | −1.2 | 0.1 | −2.3 |

*Outside ICH Q1A (R2) Guidelines. The acceptance criteria given in ICH Q1A (R2) guidelines are less than 5% change from the initial time point, therefore, the ICH acceptance criteria were met for all Formulations except 31, 34 and 36.

TABLE 36
Summary of all manufactured formulations

| | Manufact. | | | | | | Aqueous | |
|---|---|---|---|---|---|---|---|---|
| | method | OA (%) | T80 (%) | PG (%) | EtOH (%) | CBD (%) | Phase | Emuls. |
| 1 | PS | 0 | 5 | 15 | 5 | 5 | H₂O | Yes |
| 2 | PS | 5 | 5 | 11.25 | 3.75 | 5 | H₂O | Yes |
| 3 | PS | 20 | 5 | 0 | 0 | 5 | H₂O | Yes |
| 4 | PS | 20 | 5 | 0 | 0 | 5 | H₂O | Yes |
| 5 | U-T | 20 | 5 | 0 | 0 | 5 | H₂O | Yes |
| 6 | HPH | 20 | 5 | 0 | 0 | 5 | H₂O | Yes |
| 7 | HPH | 1.67 | 8.33 | 11.25 | 3.75 | 5 | H₂O | Yes |
| 8 | HPH | 0.91 | 9.09 | 11.25 | 3.75 | 5 | H₂O | Yes |
| 9 | HPH | 0.63 | 0.63 | 1.41 | 0.47 | 1.5 | H₂O | No |
| 10 | HPH | 0.63 | 0.63 | 0 | 1.88 | 1.5 | H₂O | No |
| 13 | HPH | 0.63 | 0.63 | 1.41 | 0.47 | 0.63 | H₂O | Yes |
| 14 | HPH | 1 | 1 | 1.41 | 0.47 | 1 | H₂O | Yes |
| 15 | HPH | 1.5 | 1.5 | 1.41 | 0.47 | 1.5 | H₂O | Yes |
| 16 | HPH | 1 | 0.63 | 1.41 | 0.47 | 1 | H₂O | No |
| 17 | HPH | 0.63 | 1 | 1.41 | 0.47 | 1 | H₂O | Yes |
| 18 | HPH | 1 | 1 | 1.41 | 0.47 | 1 | PBS | Yes |
| 20 | HPH | 0 | 1 | 3 | 1 | 1 | H₂O | No |
| 21 | PS | 1 | 1 | 1.41 | 0.47 | 1 | H₂O | Yes |
| 31* | HPH | 1 | 1 | 1.41 | 0.47 | 1 | H₂O | Yes |
| 32\hat{ } | HPH | 1 | 1 | 1.41 | 0.47 | 1 | H₂O | Yes |
| 33^{#} | HPH | 1 | 1 | 1.41 | 0.47 | 1 | H₂O | Yes |
| 34 | HPH | 1 | 1 | 1.41 | 0.47 | 1 | H₂O | Yes |
| 35^{$} | HPH | 1 | 1 | 1.41 | 0.47 | 1 | H₂O | Yes |
| 36^{!} | HPH | 1 | 1 | 1.41 | 0.47 | 1 | H₂O | Yes |
| 37^{&} | HPH | 1 | 1 | 1.41 | 0.47 | 1 | H₂O | Yes |

Manufacturing methods: PS—probe sonication; U-T—Ultra-Turrax; HPH—high-pressure homogenization. OA—oleic acid; T80—Tween 80; PG—pro.
Antioxidant strategy:
*= incudes 2.3 mM ascorbic acid;
\hat{ }= includes 20 mM citric acid (anhydrous).
^{#}= includes 15.8 mM sodium metabisulfite;
^{$}= includes 1.7 mM EDTA;
^{!}= includes bath sonication degassing; and
^{&}= 2.3 mM ascorbic acid and 154 mM sodium chloride.

1. A pharmaceutical composition comprising an oil-in-water emulsion having a dispersed oil phase and a continuous water phase, wherein the dispersed oil phase comprises cannabidiol (CBD) or a pharmaceutically acceptable salt or functional derivative thereof, wherein the composition is formulated for administration by inhalation.

2. The pharmaceutical composition of claim 1, wherein the dispersed oil phase comprises oil droplets having an average diameter of from about 10 nm to about 500 nm.

3. The pharmaceutical composition of claim 2, wherein one or both of the following apply:
(a) the oil droplets have an average diameter of from about 100 nm to about 300 nm; and
(b) the oil droplets have a polydispersity index (PDI) of <0.5.

4-6. (canceled)

7. The pharmaceutical composition of claim 1, comprising:
(a) from about 1 mg/mL to about 100 mg/mL CBD or a pharmaceutically acceptable salt or functional derivative thereof;
(b) from about 0.5% (w/v) to about 5% (w/v) CBD or a pharmaceutically acceptable salt or functional derivative thereof; or
(c) from about 0.5% (w/v) to about 2% (w/v) CBD or a pharmaceutically acceptable salt or functional derivative thereof.

8. (canceled)

9. (canceled)

10. The pharmaceutical composition of claim 1, wherein the dispersed oil phase comprises an oil selected from the group consisting of sesame oil, olive oil, coconut oil, medium chain triglyceride (MCT) oil and oleic acid.

11. The pharmaceutical composition of claim 10, wherein the oil is oleic acid.

12. The pharmaceutical composition of claim 1, further comprising at least one surfactant selected from the group consisting of polysorbate 80 (Tween 80), sorbitan trioleate (Span 85), disaturated-phosphatidylcholine (DSPC) and lecithin.

13. The pharmaceutical composition of claim 12, wherein the surfactant is Tween 80.

14. The pharmaceutical composition of claim 12, wherein the dispersed oil phase comprises the oil and the at least one surfactant at a ratio of from about 1:1 to about 1:10 (oil:surfactant).

15. The pharmaceutical composition of claim 14, wherein the dispersed oil phase comprises the oil and the at least one surfactant at a ratio of about 1:1 (oil:surfactant).

16. The pharmaceutical composition of claim 15, wherein the dispersed oil phase comprises the CBD or a pharmaceutically acceptable salt or functional derivative thereof, the oil and the at least one surfactant at a ratio of about 1:1:1 (CBD:oil:surfactant).

17. The pharmaceutical composition of claim 16, wherein the dispersed oil phase comprises CBD or a pharmaceutically acceptable salt or functional derivative thereof, oleic oil and Tween 80 at a ratio of about 1:1:1 (CBD:oil:surfactant).

18. The pharmaceutical composition of claim 1, further comprising one or more pharmaceutically acceptable carriers, diluents and excipients.

19. The pharmaceutical composition of claim 18, wherein the pharmaceutically acceptable excipients are selected from the group consisting of polyethylene glycol, propylene glycol, ethanol, sodium chloride, sodium citrate, tris(hydroxymethyl)aminomethane (TRIS), citric acid (anhydrous), ethylenediaminetetraacetic acid (EDTA), ascorbic acid, sodium metabilsulfite and benzalkonium chloride.

20. The pharmaceutical composition of claim 19, wherein the continuous water phase comprises an excipient selected from the group consisting of sodium metabilsulfite, EDTA, ascorbic acid, sodium chloride, citric acid and combinations of the foregoing.

21. (canceled)

22. The pharmaceutical composition of claim 1, further comprising isoflurane.

23-26. (canceled)

27. The pharmaceutical composition of claim 1, wherein said composition is formulated to be nebulized.

28. The pharmaceutical composition of claim 27, which is nebulized by a nebulizer is selected from the group consisting of a jet nebulizer, ultrasonic nebulizer, and a vibrating mesh nebulizer.

29. The pharmaceutical composition of claim 28, wherein the nebulizer is a vibrating mesh nebulizer.

30. (canceled)

31. A method for the treatment or prevention of TBI comprising administering by inhalation a therapeutically effective amount of the pharmaceutical composition of claim 1 to a subject in need thereof.

32. (canceled)
